(19) United States
(12) Patent Application Publication
(10) Pub. No.: US 2004/0087946 A1
(43) Pub. Date: May 6, 2004

(54) System and method for supporting the weight of a body using osseo-percutaneous implants

(75) Inventors: Michael Alexander Salzhauer, Surfside, FL (US); Leora S. Salzhauer, Orangeburg, NY (US)

Correspondence Address: KENYON & KENYON, 1500 K STREET, N.W., SUITE 700, WASHINGTON, DC, 20005, US

(73) Assignee: Surge-On Medical, Inc.

(21) Appl. No.: 10/284,204

(22) Filed: Oct. 31, 2002

(51) Int. Cl. A61F 4/00

(52) U.S. Cl. 606/60

(57) ABSTRACT

In accordance with embodiments of the present invention, a system for reducing pressure on high-pressure load areas on a body includes a plurality of bone anchors and a support structure. Each of the bone anchors includes an implantation end and a coupling element. The bone anchor implantation end secures the bone anchor to a load-bearing bone, while the bone anchor coupling element engages the support structure to transfer a body weight from the load-bearing bones to the support structure. The support structure may include one or more support coupling elements to engage the bone anchor coupling elements. In accordance with other embodiments of the present invention, a method for reducing pressure on high-pressure load areas on a body includes implanting the plurality of bone anchors into at least one load-bearing bone in the body, and removably attaching the coupling elements of the bone anchors to the support structure to transfer a body weight from the load-bearing bone to the support structure.

### TECHNICAL FIELD

The present invention relates to the prevention and treatment of medical conditions, e.g., pressure sores, that are caused, or exacerbated, by pressure on skin and soft tissue.

### BACKGROUND OF THE INVENTION

The prevention and treatment of pressure sores is vital to the health and well-being of debilitated patients. Pressure sores develop when localized pressure on the skin and soft tissues exceeds end-capillary pressure, causing ischemia and breakdown of local tissues. This pressure may be caused by various obvious mechanisms, such as, for example, sitting in a chair, lying on a bed, etc., for long periods of time. Various beds, mattresses, and support devices known in the art may reduce this pressure; however, none so far has been able to dramatically reduce, or eliminate, this localized pressure on skin and soft tissues.

### SUMMARY OF THE INVENTION

In accordance with embodiments of the present invention, a system for reducing pressure on high-pressure load areas on a body includes a plurality of bone anchors and a support structure. Each of the bone anchors includes an implantation end and a coupling element. The bone anchor implantation end secures the bone anchor to a load-bearing bone, while the bone anchor coupling element engages the support structure to transfer a body weight from the load-bearing bones to the support structure. The support structure may include one or more support coupling elements to engage the bone anchor coupling elements.

In accordance with other embodiments of the present invention, a method for reducing pressure on high-pressure load areas on a body includes implanting the plurality of bone anchors into at least one load-bearing bone in the body, and removably attaching the coupling elements of the bone anchors to the support structure to transfer a body weight from the load-bearing bone to the support structure.

### BRIEF DESCRIPTION OF THE DRAWINGS

FIG. 1 is a schematic representation of a bone anchor, according to an embodiment of the present invention.

FIG. 2 depicts a plurality of bone anchors attached to a load-bearing bone and a corresponding support structure, according to an embodiment of the present invention.

FIG. 3 suggests various bone anchor implantation locations.

FIG. 4 depicts a wheelchair support structure for reducing pressure on high-pressure load areas on a body, according to an embodiment of the present invention.

FIG. 5 depicts a bed support structure for reducing pressure on high-pressure load areas on a body, according to an embodiment of the present invention.

FIG. 6 depicts another support structure for reducing pressure on high-pressure load areas on a body, according to an embodiment of the present invention.

FIG. 7 shows a method for reducing pressure on high-pressure load areas on a body, according to an embodiment of the present invention.

### DETAILED DESCRIPTION

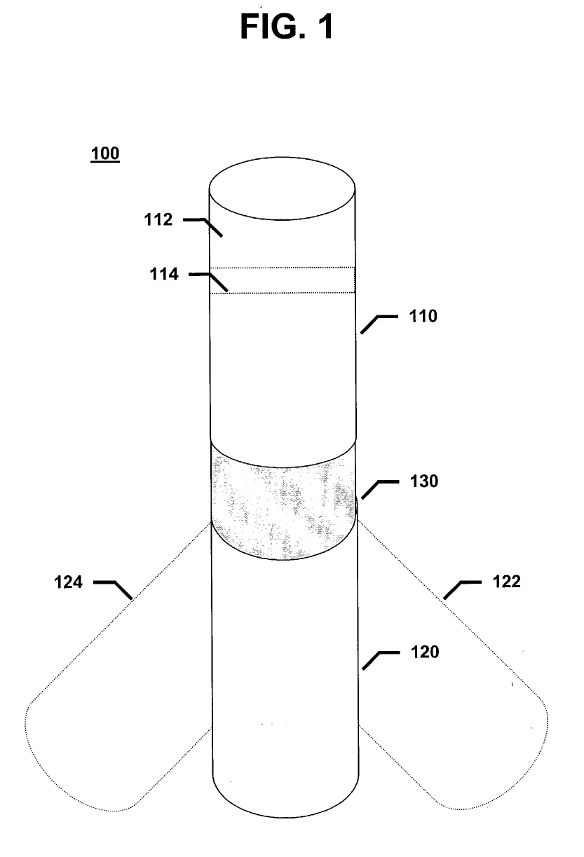

FIG. 1 is a schematic representation of a bone anchor according to an embodiment of the present invention. Bone anchor 100 may be constructed of any suitable implant material, such as, for example, surgical stainless steel, titanium, etc., and should withstand at least a steady-state, longitudinal compression load in the normal range of human weights, such as, for example, 0 Kg to 200 Kg, plus an optional factor of safety. Generally, the physical dimensions (e.g., length, diameter, cross-sectional area, etc.) of bone anchor 100 are selected to support this steady-state, longitudinal compression load, as well as to withstand any dynamic loading attendant with the system. Bone anchor 100 should also withstand various loads applied in the transverse, or radial, direction, such as, for example, shear forces, bending moments, torques, etc. These forces may be applied to bone anchor 100, for example, during implantation, during the engagement of bone anchor 100 with a support structure, etc.

In one embodiment, bone anchor 100 may be cylindrical, with a cross-sectional area generally circular along its length. In other embodiments, different cross-sectional geometries, such as hexagonal, rectangular, square, etc., may be used. These cross-sectional geometries may also vary, in shape or dimensions, along the length of bone anchor 100. Accordingly, the cross-sectional geometry may be optimized based on various design considerations, such as, for example, force and moment distribution, implant location, internal obstructions, etc. The bone anchor may include an antibiotic material coating to promote long-term implantation without disruption. Materials may be impregnated in the bone anchor to promote osseointegration. Examples of materials that may be used on, or within, the bone anchor include keratin, collagen, etc.

Bone anchor 100 includes implantation end 110 to facilitate the implantation of bone anchor 100 into a load-bearing bone. In an embodiment, surface 112 of implantation end 110 may be smooth, while in another embodiment, surface 112 of implantation end 110 may be rough, or textured, to promote osseointegration. In a further embodiment, implantation end 110 may include at least one transverse hole 114 (shown in phantom outline) to promote osseointegration. At its other end, bone anchor 100 includes coupling element 120 to engage the support structure. After implantation and engagement, the body weight is transferred from the load-bearing bone to the support structure. In one embodiment, bone anchor 100 may be one contiguous member, while in another embodiment, bone anchor 100 may include at least two separate members, e.g., one member having implantation end 110 and another member having coupling element 120. In the latter embodiment, implantation end 110 and coupling element 120 may be removably attached at attachment region 130. Coupling element 120 may be removably attached to implantation end 110 before implantation, or, alternatively, coupling element 120 may be removably attached to implantation end 110 after implantation.

In a further embodiment, bone anchor 100 may include multiple coupling elements to engage at least one corresponding support coupling element of the support structure. For example, bone anchor 100 may include implantation end 110, coupling element 120, coupling element 122 (shown in phantom outline) and coupling element 124 (shown in phantom outline). In this embodiment, coupling elements 120, 122 and 124 may simultaneously engage multiple corresponding support coupling elements of the support structure. Alternatively, depending upon the orientation of the body, only one of the coupling elements 120, 122 or 124 may engage in corresponding support coupling element of the support structure. For example, to support the body in a sitting position, coupling element 120 may be engaged to a corresponding support coupling element, while coupling elements 122 and 124 are not engaged. Similarly, to support the body in a supine position, coupling element 122 may be engaged to a corresponding support coupling element, while coupling elements 120 and 124 are not engaged. Finally, to support the body in a prone position, coupling element 124 may be engaged to a corresponding support coupling element, while coupling elements 120 and 122 are not engaged.

Coupling element 120 may be interchanged with a different coupling element after implantation. In this manner, coupling element 120 may be replaced by a new coupling of the same type. Alternatively, coupling element 120 may be replaced by a new coupling of a different type in order to correspond to the particular support structure. Various well-known coupling mechanisms may be used to removably attach implantation end 110 to coupling element 120 at attachment region 130, such as, for example screw-thread couplings, sleeve couplings, insert couplings, jaw couplings, key-keyway couplings, etc. In one example, implantation end 110 may include tap threads while coupling element 120 may include corresponding screw threads adapted to engage the tap threads to removably attach the two members.

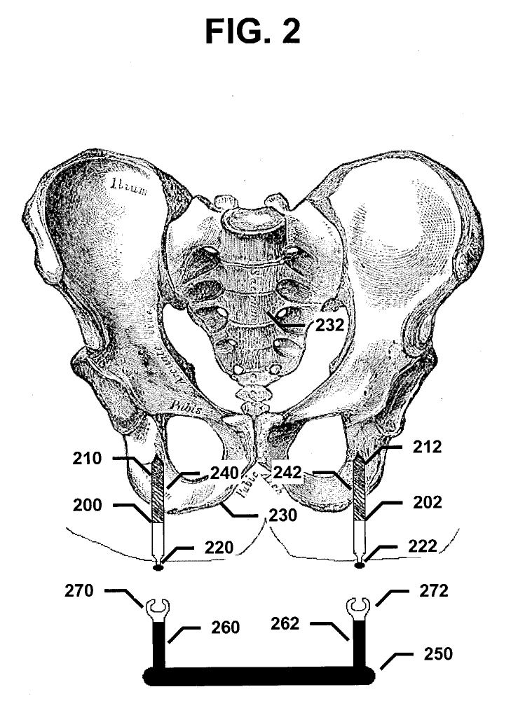

FIG. 2 depicts a plurality of bone anchors attached to a load-bearing bone, according to an embodiment of the present invention. In this illustrated embodiment, implantation ends 210 and 212 of bone anchors 200 and 202, respectively, may include screw threads. For example, holes 240 and 242 may be drilled and tapped into ischium 230, and then bone anchors 200 and 202 may be screwed into holes 240 and 242, respectively. In another embodiment, implantation ends 210 and 212 of bone anchors 200 and 202, respectively, may include self-tapping screw threads, and holes 240 and 242 may be drilled, but not tapped, into ischium 230, i.e., holes 240 and 242 may include a smooth bore. In this embodiment, holes 240 and 242 may be tapped as bone anchors 200 and 202 are screwed into holes 240 and 242, respectively, for example. Various other well-known mechanisms may be used to attach bone anchors 200 and 202 to a load-bearing bone, and specifically, for example, to ischium 230.

Advantageously, an adapter (not shown) may be attached to bone anchor 200, e.g., to coupling element 220, to facilitate implantation of bone anchor 200 into a load-bearing bone. Similarly, an adapter may be attached to bone anchor 202 to facilitate implantation. In one embodiment, the adapter may include a lever and a catch, adapted to engage a latch, on coupling element 220, to transfer a turning moment, or torque, from the lever, through the catch and latch, to bone anchor 200. Accordingly, bone anchor 200 may be screwed into ischium 230, for example, through rotation of the lever. In another embodiment, a screw head may be attached to coupling element 220, so that a screwdriver, power screwdriver or drill may be used to screw bone anchor 200 into ischium 230. Other well-known mechanisms may also be used to implant bone anchor 200 into a load-bearing bone.

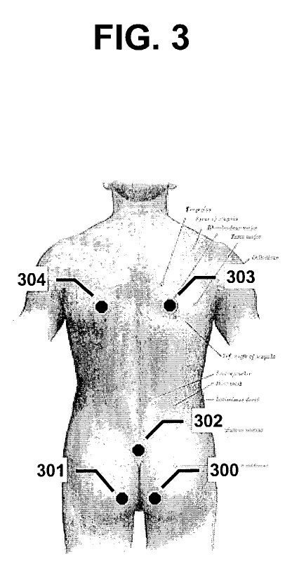

FIG. 3 suggests various locations for a plurality bone anchors. Typically, at least two bone anchors may be used to reduce pressure on the high-pressure areas of the body, as indicated, for example, by locations 300, 301, 302, 303, 304, etc. For a body in a sitting position, two bone anchors may be sufficient to reduce this pressure, while for a body in a supine position, three or four bone anchors may be required. In the latter example, additional attachment points reduce the transferred load at any one point, which may be advantageous in those locations where the thickness of the load-bearing bone may be smaller. Various locations 300, 301, 302, 303, 304, etc., are depicted in FIG. 3. Other locations may be used to support the body in various orientations, such as, for example, lateral, prone, ¾ lateral, etc. While FIG. 3 depicts a human body, animals may similarly benefit from the present invention.

Referring again to FIG. 2, bone anchors 200 and 202, when implanted, protrude through a section of skin proximal to the load-bearing bone. For example, bone anchors 200 and 202 may be attached to ischium 230 and may protrude through the soft tissues, gluteus maximus and surrounding epidermal layer. Bone anchors 200 and 202 may extend through the skin sufficiently to expose anchor coupling elements 220 and 222 for engagement with support structure 250.

As depicted in FIG. 2, support structure 250 may include a plurality of support posts, such as, for example, support posts 260 and 262. Located at the ends of these support posts are support coupling elements to engage the anchor coupling elements, such as, for example, support coupling elements 270 and 272. Anchor coupling elements 220 and 222 may include any number of well-known mechanisms to engage, and to be supported by, support structure 250. For example, in one embodiment, anchor coupling elements 220 and 222 may comprise ball joints to be mated with corresponding sockets of support coupling elements 270 and 272, respectively. Conversely, anchor coupling elements 220 and 222 may comprise sockets to be mated with corresponding ball joints of support coupling elements 270 and 272. In another embodiment, anchor coupling elements 220 and 222 may comprise pegs to be mated with corresponding prongs of support coupling elements 270 and 272. Conversely, anchor coupling elements 220 and 222 may comprise prongs to be mated with corresponding pegs of support coupling elements 270 and 272. In an embodiment, the anchor coupling elements (e.g., prongs) may be removably attached to bone anchors using, for example, a screw-thread mechanism. After the bone anchors are implanted, the anchor coupling elements may be detached, e.g., screwed off, and the penetration through the skin closed over. In a further embodiment, anchor coupling elements 220 and 222 may comprise general latch mechanisms to be mated with corresponding catch mechanisms of support coupling elements 270 and 272. Conversely, anchor coupling elements 220 and 222 may comprise general catch mechanisms to be mated with corresponding latch mechanisms of support coupling elements 270 and 272.

In another embodiment, anchor coupling elements 220 and 222 may be constructed of, or may include, magnetic material to magnetically engage, in opposition, corresponding magnets, or electromagnets, of support coupling elements 270 and 272. In this embodiment, there may not be physical contact between anchor coupling elements 220, 222 and support coupling elements 270, 272. Rather, opposing magnetic fields, produced by anchor coupling elements 220, 222 and support coupling elements 270, 272, levitate the body above support structure 250. In this manner, the anchor coupling elements magnetically engage support structure 250. In an alternative embodiment, supporting posts 260 and 262 may be replaced, and the support coupling element may comprise a magnet, electromagnet, group of magnets, or group of electromagnets, contained within support structure 250. It will be appreciated that a single structure may be used as a support coupling element to support more than one bone anchor. Thus, a single magnet or electromagnet may magnetically engage and support multiple bone anchors. Similarly, a single rod or cross bar may be used as a support coupling element to engage multiple bone anchor coupling elements. In another embodiment, anchor coupling elements 220 and 222 may be contained within the body and may not extend through the skin. Advantageously, magnetic levitation may also provide a degree of shock-absorption.

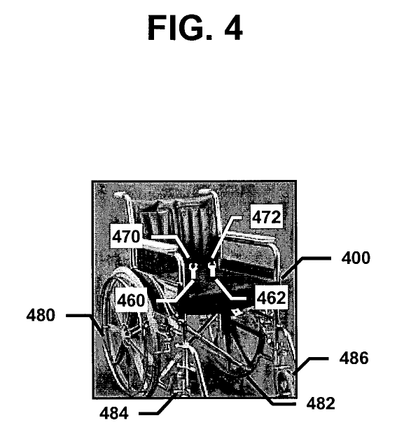

The coupling mechanism used to engage anchor coupling elements 220 and 222 to support coupling elements 270 and 272, respectively, may provide for rapid disengagement. In other words, when bone anchors 200 and 202 are engaged to support structure 250, bone anchors 200 and 202 may be disengaged in a rapid manner to avoid inflicting additional injury in an emergency situation. With reference to FIG. 4, for example, anchor coupling elements 220 and 222 may disengage rapidly from support coupling elements 270 and 272 when support structure 250 experiences a sudden impact or overturning moment (e.g., a wheelchair tumbling over).

FIG. 4 depicts an embodiment in which the support structure for reducing pressure on high-pressure load areas on a body is a specially-configured wheelchair. Thus, in the embodiment illustrated in FIG. 4, support structure 400 is the wheelchair itself, and support posts 460 and 462, with support coupling elements 470 and 472, may be attached to the wheelchair's frame, for example. In another embodiment, support structure 400 may be a separate unit, or frame, to which the support posts and support coupling elements may be attached. In this embodiment, the separate unit, or frame, may be fixedly, or removably, attached to the wheelchair.

In the FIG. 4 embodiment, several support coupling elements may be attached to support structure 400. In this embodiment, support posts 460 and 462 are attached to support structure 400, and include support coupling elements 470 and 472, respectively. As illustrated in FIG. 4, support structure 400 may support the body in a sitting position. In this illustrated embodiment, the number of bone anchors attached to load-bearing bones within the body may be complementary to the number of supporting elements attached to support structure 400. Thus, as illustrated, two support posts and support coupling elements are provided and, consequently, two bone anchors should be implanted in the appropriate locations, such as, for example, implantation locations 300 and 301. As discussed with reference to FIG. 2, for example, support coupling elements 470 and 472 may be sockets to engage ball joints on the corresponding bone anchor coupling elements. In other examples, support coupling elements 470 and 472 may be prongs to engage pegs on the corresponding bone anchor coupling elements, catches to engage latches, etc. The converse orientations may also be used.

The support coupling elements 470 and 472 may be removably attached to support posts 460 and 462, respectively, to allow for different types of coupling elements to be provided. In another embodiment, the support posts 460 and 462 may be removable attached to the remainder of support structure 400 to allow for various numbers, or different types, of supporting elements. In a further embodiment, shock-absorption devices (not shown) may interpose between support posts 460, 462 and a frame (not shown) within support structure 400, to reduce the forces attendant with engaging (e.g., sitting on) support posts 460 and 462. Shock-absorbers may also cushion the ride, since support structure 400 is mobile and includes main wheels 480 and 482, and guide wheels 484 and 486.

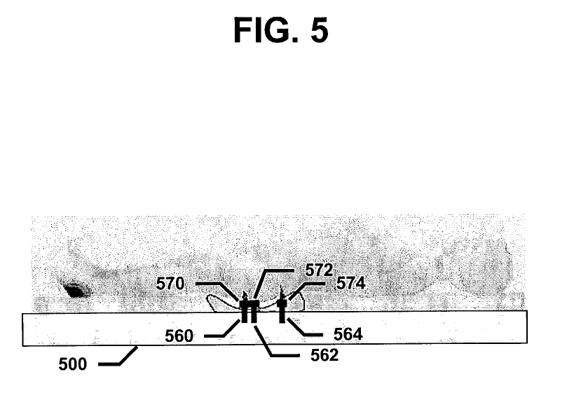

FIG. 5 depicts an embodiment in which the support structure for reducing pressure on high-pressure load areas on a body is a specially-configured bed. Thus, in the embodiment illustrated in FIG. 5, support structure 500 is the bed itself, and support posts 560, 562 and 564, with support coupling elements 570, 572 and 574, may be attached, for example, to the bed's frame. In another embodiment, support structure 500 may be a separate unit, or frame, to which the support posts and support coupling elements may be attached. In this embodiment, the separate unit, or frame, may be fixedly or removably attached to the bed.

In the FIG. 5 embodiment, several supporting elements may be attached to support structure 500. In this embodiment, support posts 560, 562 and 564 are attached to support structure 500, and include support coupling elements 570, 572 and 574, respectively. As illustrated in FIG. 5, support structure 500 may support the body in a supine position. Again, in this illustrated embodiment, the number of bone anchors attached to load-bearing bones within the body may be complementary to the number of supporting elements attached to support structure 500. Thus, as illustrated, three support posts and support coupling elements are provided, and, consequently, three bone anchors should be implanted in the appropriate locations, such as, for example, implantation locations 300, 301 and 302. As discussed with reference to FIG. 2, for example, support coupling elements 570, 572 and 574 may be sockets to engage ball joints on the corresponding bone anchor coupling elements. In other examples, support coupling elements 570, 572 and 574 may be prongs to engage pegs on the corresponding bone anchor coupling elements, catches to engage latches, etc. The converse orientations may also be used.

The support coupling elements 570, 572 and 574 may be removably attached to support posts 560, 562 and 564, respectively, to allow for different types of coupling elements to be provided. In another embodiment, support posts 560, 562 and 564 may be removable attached to the remainder of support structure 500 to allow for various numbers, or different types, of supporting elements. In a further embodiment, shock-absorption devices (not shown) may interpose between support posts 560, 562, 564 and a frame (not shown) within support structure 500 to reduce the forces attendant with engaging (e.g., lying on) support posts 560, 562 and 564. Shock-absorbers may also cushion the ride, if support structure 500 includes wheels.

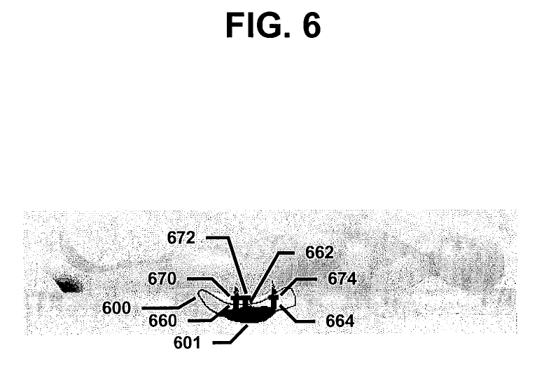

FIG. 6 depicts another support structure for reducing pressure on high-pressure load areas on a body, according to an embodiment of the present invention. In this embodiment, support structure 600 includes support posts 660, 662 and 664 and support coupling elements 670, 672 and 674. As illustrated in FIG. 6, support structure 600 may support the body in a supine position. In this illustrated embodiment, three support posts and support coupling elements are provided, and, consequently, three bone anchors should be implanted in the appropriate locations, such as, for example, implantation locations 300, 301 and 302. As discussed with reference to FIG. 2, for example, support coupling elements 670, 672 and 674 may be sockets to engage ball joints on the corresponding bone anchor coupling elements. In other examples, support coupling elements 670, 672 and 674 may be prongs to engage pegs on the corresponding bone anchor coupling elements, catches to engage latches, etc. The converse orientations may also be used.

The support coupling elements 670, 672 and 674 may be removably attached to support posts 660, 662 and 664, respectively, to allow for different types of coupling elements to be provided. In another embodiment, support posts 660, 662 and 664 may be removably attached to the remainder of support structure 600 to allow for various numbers, or different types, of supporting elements. In a further embodiment, shock-absorption devices (not shown) may interpose between support posts 660, 662, 664 and a frame (not shown) within support structure 600 to reduce the forces attendant with engaging (e.g., lying on) support posts 660, 662 and 664. Shock-absorbers may also cushion the ride, since support structure 600 may be mobile and may include an interface 601. In one embodiment, interface 601 may be a rolling ball-in-socket, such as, for example, a structure similar to a computer mouse, while in another embodiment, interface 601 may be a low-friction surface. In a further embodiment, interface 601 may include a set of wheels.

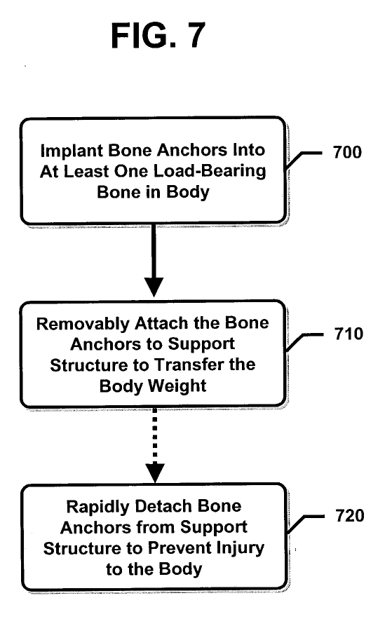

FIG. 7 shows a method for reducing pressure on high-pressure load areas on a body, according to an embodiment of the present invention. A plurality of bone anchors may be implanted (700) into at least one load-bearing bone in the body. The bone anchors have an implantation end and a coupling element, as discussed above with reference to FIGS. 1 and 2. For example, in a sitting embodiment, two bone anchors may be implanted (700) into ischium 230, such as, for example, bone anchors 200 and 202. Generally, for the sitting embodiment, two bone anchors 200 and 202 may be located at implantation locations 300 and 301. Other body orientation embodiments may require more bone anchors, different implantation locations, etc. For example, in a supine embodiment, two bone anchors may be implanted (700) into ischium 230, and one bone anchor may be implanted (700) into sacram 232, corresponding, generally, to implantation locations 300, 301 and 302.

The bone anchors may then be removably attached (710) to a support structure to transfer the body weight from the load-bearing bone to the support structure. The support structure may have several support posts, each having a support coupling element, to engage the bone anchor coupling elements, as discussed above with reference to FIGS. 2 and 4-6. For example, in the sitting embodiment, the support structure may have two support posts, such as, for example, support posts 260 and 262. Generally, support coupling elements 270 and 272 complement bone anchor coupling elements 220 and 222, and may be removably attached (710) together. Other body orientation embodiments may require more support posts and support coupling elements in appropriate sections of the support structure. For example, in the supine embodiment, three support posts and support coupling elements may be provided within support structure 500, corresponding, generally, to bone anchors implanted in implantation locations 300, 301 and 302.

If desired, the anchor coupling elements and the support coupling elements may be adapted to be rapidly detached (720) to prevent injury to the body. For example, in the sitting embodiment, anchor coupling elements 220 and 222 may be adapted to be rapidly detached from support coupling elements 270 and 272 if support structure 250, in the form of a wheelchair (i.e., support structure 400), experiences a sudden overturning moment.

Several embodiments of the present invention are specifically illustrated and described herein. However, it will be appreciated that modifications and variations of the present invention are covered by the above teachings and within the purview of the appended claims without departing from the spirit and intended scope of the invention.

What is claimed is:

1. A system to reduce pressure on high-pressure load areas on a body, comprising:
a support structure; and
a plurality of bone anchors, each of the plurality of bone anchors including:
an anchor implantation end to secure the bone anchor to a load-bearing bone, and
at least one anchor coupling element adapted to engage the support structure to transfer a body weight from the load-bearing bone to the support structure.

2. The system of claim 1, wherein each bone anchor protrudes through a section of skin proximal to the load-bearing bone such that each anchor coupling element is located outside the body after the bone anchor is attached to the load-bearing bone.

3. The system of claim 1, wherein the anchor coupling element of at least one bone anchor is adapted to be removably attached to the remainder of the bone anchor.

4. The system of claim 1, wherein the plurality of bone anchors include antibiotic material.

5. The system of claim 1, wherein:
the support structure includes at least one support coupling element, and
each support coupling element is adapted to engage at least one anchor coupling element.

6. The system of claim 5, wherein at least one anchor coupling element comprises a ball and at least one support coupling element comprises a socket.

7. The system of claim 5, wherein at least one support coupling element comprises a ball and at least one anchor coupling element comprises a socket.

8. The system of claim 5, wherein at least one anchor coupling element comprises a peg and at least one support coupling element comprises a prong.

9. The system of claim 5, wherein at least one support coupling element comprises a peg and at least one anchor coupling element comprises a prong.

10. The system of claim 9, wherein the prong of at least one bone anchor is adapted to be removably attached to the remainder of the bone anchor.

11. The system of claim 5, wherein at least one anchor coupling element comprises a latch and at least one support coupling element comprises a catch.

12. The system of claim 5, wherein at least one support coupling element comprises a latch and at least one anchor coupling element comprises a catch.

13. The system of claim 5, wherein at least one anchor coupling element comprises magnetic material and at least one support coupling element comprises a magnet to magnetically engage the anchor coupling elements.

14. The system of claim 5, wherein at least one support coupling element comprises magnetic material and at least one anchor coupling element comprises a magnet to magnetically engage the anchor coupling elements.

15. The system of claim 5, wherein the support structure includes at least one shock-absorber attached to at least one support coupling element.

16. The system of claim 5, wherein at least one support coupling element includes a rod.

17. The system of claim 1, wherein the system is adapted to elevate the body above the support structure.

18. The system of claim 1, wherein the system is adapted to suspend the body below the support structure.

19. The system of claim 1, wherein the support structure comprises a bed.

20. The system of claim 1, wherein the support structure comprises a wheelchair.

21. The system of claim 1, wherein the support structure comprises a walker.

22. The system of claim 1, wherein the support structure includes a rolling ball-in-socket element.

23. The system of claim 1, wherein the support structure includes a low-friction surface.

24. A system to reduce pressure on high-pressure load areas on a body, comprising:
a support structure; and
a plurality of bone anchors, each of the plurality of bone anchors including:
means for securing the bone anchor to a load-bearing bone, and
means for coupling the bone anchor to the support structure to transfer a body weight from the load-bearing bone to the support structure.

25. A method for reducing pressure on high-pressure load areas on a body, comprising:
providing a support structure and a plurality of bone anchors;
implanting the plurality of bone anchors into at least one load-bearing bone in the body, each bone anchor having an anchor implantation end and an anchor coupling element; and
removably attaching the anchor coupling elements to the support structure to transfer a body weight from the load-bearing bone to the support structure.

26. The method of claim 25, wherein the anchor coupling elements are adapted to be detached rapidly from the support structure to prevent injury to the body.
